(19) United States
(12) Patent Application Publication
(10) Pub. No.: US 2026/0108632 A1
(43) Pub. Date: Apr. 23, 2026

(54) METHOD FOR PRODUCING A SPONTANEOUS METASTASIS MODEL

(71) Applicants: MESTASTOP SOLUTIONS PRIVATE LIMITED, Bangalore (IN); IMMUNOBIOME INC., Pohang (KR)

(72) Inventors: Amit SHARMA, Pohang (KR); Debabani Roy CHOWDHURY, Bangalore (IN); Garima SHARMA, Pohang (KR); Samrat ROY, Bangalore (IN); Manoj PANDRE, Bangalore (IN); Soo-Woong LEE, Pohang (KR); ChangWook PARK, Seoul (KR); Rajesh KUMAR RK, HOSUR (IN); Sundarajan KANNAN, BANGALORE (IN); Arnab Roy CHOWDHURY, Bangalore (IN)

(21) Appl. No.: 18/292,945

(22) PCT Filed: Oct. 18, 2022
(86) PCT No.: PCT/IN2022/050928

§ 371 Date: Jan. 29, 2024

(30) Foreign Application Priority Data
Dec. 30, 2021 (IN) 202141061794

(51) Int. Cl. A61K 49/00; A01K 67/0271; A61P 35/04; C12N 15/85

(52) U.S. Cl. CPC A61K 49/0008; A01K 67/0271; A61P 35/04; C12N 15/85; A01K 2207/12; A01K 2207/15; A01K 2227/105; A01K 2267/0331

(57) ABSTRACT

Method for producing a spontaneous metastasis model is disclosed herein. The models, according to various embodiments herein, achieve a higher metastasis incidence rate, in an instance about 100% for mesenteric lymph node (mLN) invasion and about 80 to 90% for secondary organs. The models are kinetic model having a luciferase-based expression system that provide more statistically significant and robust data. The embodiments herein further include a method for producing the model for metastasis, comprising transplanting recombinant carcinoma cells having increased invasiveness and decreased tumorigenic properties and luciferase gene expression system, in one or more orthotopic positions in an animal.

### CROSS REFERENCE TO RELATED APPLICATIONS

This application is based on and derives the benefit of Indian Application IN 202141061794 filed on Dec. 30, 2021, the contents of which are incorporated herein by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 11,000 Byte XML file named “Sequence-listing.xml,” dated Dec. 26, 2024.

### TECHNICAL FIELD

The present invention relates to cancer metastasis, and more particularly to models for cancer metastasis and uses thereof. It further relates to methods for producing the cancer metastasis models.

### BACKGROUND

Metastasis, the spread of malignant cells from a primary tumor to distant sites, is responsible for about 90% of cancer-related deaths. Using in vitro cell culture and in vivo animal models, significant progress has been made to understand the genetic and cellular aspects of metastasis. However, successful bench-to-bedside translation of the experimental models is very crucial for finding appropriate therapeutic interventions.

Different animal models have been designed to study cancer metastasis. They include experimental and spontaneous metastasis models which can be developed as syngeneic or human xenografts in mice. The experimental models of metastasis are fast but have certain shortcomings. Experimental models do not recapitulate the establishment of primary tumor formation as the tumor cells are directly implanted into the blood. The syngeneic models involve the use of murine tumor cells and therefore lack complete human relevance. Based on implantation of tumor cells, spontaneous metastasis models are further divided into heterotopic and orthotopic. Tumor cells implanted subcutaneously, heterotopic models, in mice generally tend to grow rapidly and thus do not represent the slower doubling times of most of the human cancers.

Studies using orthotopic models, tissue-specific tumor implantation, represent clinical trial metastasis patients better as they recapitulate metastasis by enabling interactions with tissue of origin, initial invasion and distant metastatic spread. Orthotopic metastasis models have been successful for a number of tumor types including mammary, pancreatic, lung and colon. However, these metastasis models generally require long experimentation periods (4 to 6 months), a copious use of animals and are endpoint studies thereby increasing overall cost of study.

### OBJECTS

The principal object of the embodiments disclosed herein is to provide a spontaneous model for cancer metastasis.

A second object of the embodiments disclosed herein is to provide an animal model having low latency for metastasis and higher incidence rate.

An object of the embodiments disclosed herein is to provide an orthotopic animal model for spontaneous metastasis, comprising recombinant primary tumor cells having reduced tumorigenicity and increased invasiveness.

An object of the embodiments disclosed herein is to provide animal model for spontaneous metastasis that are biologically relevant and capable of exhibiting early metastasis, for e.g.: as early as within a period of about 6 weeks.

Another object of the embodiments disclosed herein is to provide a metastasis model capable of kinetic evaluation, without the requirement of animal sacrifice.

Another object of the embodiments disclosed herein is to provide an animal model for metastasis capable of providing data which is statistically significant and robust.

An object of the embodiments disclosed herein is to provide an animal model for evaluating liver metastasis of colorectal cancer.

An object of the embodiments disclosed herein is to provide a kinetic animal model for screening or evaluating pharmaceutical substances, for e.g.: New Chemical Entities (NCE) and other potential therapeutic candidates including chemical drugs, biotherapeutics, biosimilars, etc, for anti-cancer or anti-metastatic properties.

Another object of the embodiments disclosed herein is to provide an animal model for evaluating drug repurposing, particularly for their potential use in preventing or inhibiting cancer metastasis.

An object of the embodiments disclosed herein is to provide a method for screening or evaluating pharmaceutical substances, for e.g.: New Chemical Entities (NCE) and other potential therapeutic candidates including chemical drugs, biotherapeutics, biosimilars, etc, for anti-cancer or anti-metastatic properties.

Another object of the embodiments disclosed herein is to provide a method for evaluating drug repurposing, particularly for their potential use in inhibiting cancer metastasis.

Another object of the embodiments disclosed herein is to provide an animal model for evaluating anti-cancer therapy, particularly for their potential use in inhibiting cancer metastasis.

Another object of the embodiments disclosed herein is to provide a method for evaluating anti-cancer therapy, particularly for their potential use in inhibiting cancer metastasis.

An object of the embodiments disclosed herein is to provide a method for producing the animal model of spontaneous metastasis, as disclosed in various embodiments herein.

Another object of the embodiments disclosed herein is to provide a method for evaluating metastasis using the animal model of spontaneous metastasis.

Another object of the embodiments disclosed herein is to provide a cost-effective and time sensitive method for evaluating metastasis and/or cancer.

An object of the embodiments disclosed herein is to provide a method for evaluating liver metastasis of colorectal cancer.

An object of the embodiments disclosed herein is to provide a method for accelerating metastasis in an animal model of cancer.

These and other objects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF DRAWINGS

The embodiments disclosed herein are illustrated in the accompanying drawings. The embodiments herein will be better understood from the following description with reference to the drawings, in which:

FIG. 1 is a schematic representation of vectors containing gene of interest (GOI) and selectable marker used in the study, wherein 1(a) depicts a constitutive vector; and 1(b) depicts an inducible vector, according to embodiments disclosed herein;

FIG. 2 is a flowchart illustrating different strategies for constructing cells with high Plasticity Ratio (PR), and producing the model, according to embodiments disclosed herein;

FIG. 3 is a schematic representation of luciferase encoding vectors, wherein 3(a) depicts a lentiviral vector (VB2014-1486aax); and 3(b) depicts a plasmid vector (VB210920), according to embodiments disclosed herein;

FIG. 4(a) is a graph depicting flow cytometry analysis of HT29 clone 12BC6 (inducible; clone 1) and HT29 clone 8C5 (constitutive; clone 2) for expression of Snail, Vimentin, E-Cadherin, CD 133, and CD 44 proteins;

FIG. 4(b) is a western blot gel depicting protein expression for Tubulin (loading control) and SNAIL protein in COLO-205, HCT-116, HT-29, SW-480, HT29 clone 8C5 and HT29 clone 12BC6 cell lines;

FIG. 5 are graphical representations depicting tumor volume at different number of cells transplanted in NOD-SCID mice (0.5, 1, & 5 million/mouse respectively), wherein 5(a) illustrates a comparison of tumor volume in HT29 WT cells (with PR ratio of 0.4) and HT29 8C5 cells (constitutive clone of HT29 with PR ratio of 0.85); while 5(b) illustrates a comparison of tumor size in SW480 WT cells (with PR ratio of 1.1) and SW480 1C3 (constitutive clone of SW480 with PR ratio of 1.3).

FIG. 6 are pictorial images depicting tumor size; wherein left, center and right images correspond to number of cells transplanted in NOD-SCID mice (0.5, 1, & 5 million/mouse respectively); 6(a) illustrates tumor size in NOD-SCID mice injected with HT29 WT cells; 6(b) illustrates tumor size in NOD-SCID mice injected with HT29 8C5 cells; and 6(c) illustrates tumor size in NOD-SCID mice injected with SW480 WT cells;

FIG. 7 is a graph depicting effect of luciferase on tumor volume in NOD-SCID mice injected with different cells: HT29_WT, HT29_WT_Luc, HT29 8C5, HT29 8C5_Luc, respectively;

FIG. 8(a) are bioluminescence images depicting NOD-SCID mice injected with HT29-12BC6_Luc cells with different strategies for induction i.e., Tet on, Tet off-on, and Tet off-on-off at day 6 and day 8 respectively, post tumor cell implantation and with differing schemes for tetracycline mediated high PR induction;

FIG. 8(b) is a graph depicting bioluminescence in livers of NOD-SCID mice injected with HT29-12BC6_Luc Tet on cells, HT29-12BC6_Luc Tet off-on cells and HT29-12BC6_Luc Tet off-on-off cells respectively, as observed till day 40;

FIG. 9 are bioluminescence images depicting mesenteric lymph nodes (mLN) isolated from NOD-SCID mice injected with HT29-12BC6_Luc Tet on cells, HT29-12BC6_Luc Tet off-on cells and HT29-12BC6_LucTet-off-on-off cells (An #1, An #2, An #3, An #4, and An #5 represent animal number 1, 2, 3, 4, and 5 respectively);

FIG. 10 is a graph depicting relative bioluminescence intensity over liver in mice, orthotopically injected with HT29 WT Luc and HT29-12BC6_Luc cells;

FIG. 11(a) is a graph depicting liver bioluminescence intensity in animals with orthotopic implantation of HT29-12BC6_Luc cells and treated with MS-AP-003; and

FIG. 11(b) is a graph depicting liver bioluminescence intensity in animals with orthotopic implantation of HT29-12BC6_Luc cells and treated with AP-006.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as not to unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein disclose models for spontaneous metastasis and method for production thereof. The embodiments disclosed herein, include the use of recombinant tumor cells produced by converting non-metastatic (non-met) cells to metastatic (met) cells, having increased invasiveness and reduced tumorigenicity. The models, according to embodiments herein, have lower latency period for metastasis, in an instance about 4 to 6 weeks, due to increased invasiveness of recombinant primary tumor cells. The models, according to various embodiments herein, achieve a higher metastasis incidence rate, in an instance about 100% for mesenteric lymph node (mLN) invasion and about 80-90% for secondary organs. The models are kinetic model having a luciferase-based expression system that provide more statistically significant and robust data. Thus, tumor progression can be observed via live-cell imaging, avoiding the need for sacrificing animals. In some cases, tumor growth may be visualized as early as a week. At about 6 weeks, 80-100% of the animals show metastasis using the disclosed model. The model also includes a stringency feature, wherein inducible vector may be controlled by administering the mice with a diet that includes doxycycline. Animals carrying luciferase expressing cancer cells may be visualized by administering luciferin, preferably by intraperitoneal injection of luciferin. Overall, the embodiments herein disclose a model that is improved, cost-effective and time sensitive.

The embodiments herein further include a method for producing the model for metastasis, comprising transplanting recombinant carcinoma cells having increased invasiveness and decreased tumorigenic properties and Luciferase gene expression system, in one or more orthotopic positions in an animal. The recombinant cells, according to embodiments herein, are screened and selected based on phenotypic properties, particularly plasticity ratio (PR). In an embodiment, cancer cell lines are genetically modified to exhibit higher PR as compared to wild type cancer cells or cell lines without modifications.

The term “plasticity”, as used herein refers to the ability of cells to transition into one or more cell types, for e.g.: epithelial cell transition into mesenchymal cell, and vice versa. Typically, cancer cells are known to exhibit epithelial plasticity during cancer metastasis, wherein the cells transition from epithelial-like characteristics to mesenchymal-like characteristics. Such transitions in cell phenotype from non-motile epithelial to motile mesenchymal forms, or vice versa, is generally known as epithelial to mesenchymal transitions (EMT) or mesenchymal to epithelial transition (MET) respectively. The markers, according to embodiments herein, include markers that are associated with epithelial to mesenchymal transitions or mesenchymal to epithelial transitions. In an embodiment, the markers are epithelial markers. In another embodiment, the markers are mesenchymal markers. In an embodiment, the markers are epithelial and mesenchymal markers associated with epithelial to mesenchymal transitions. Examples of epithelial markers include, but are not limited to, Collagen IV alpha 1, Cytokeratin (pan), Desmoglein-3, E-Cadherin, Laminin, MUC-1, Syndecan-1, EpCAM etc. Examples of mesenchymal markers include, but are not limited to, Alpha-SMA, Fibronectin, N-Cadherin, S100A4, Vimentin, etc. The markers, according to embodiments herein include, but are not limited to, N-Cadherin, Vimentin, E-cadherin and EpCam. In an embodiment, the mesenchymal markers include N-Cadherin, and Vimentin. In an embodiment, the epithelial markers include E-Cadherin and EpCAM.

N-Cadherin and E-Cadherin are type-I classical cadherins that are calcium dependent membrane proteins forming adherence junctions. E cadherin is found in epithelial tissue and N cadherin is prevalent in non-epithelial tissues. Vimentin, a type III member of intermediate filament family of proteins, is found in animal cells and ubiquitously expressed in normal mesenchymal cells. Overexpression of Vimentin has been associated with various epithelial cancers. EpCam (Epithelial Cell Adhesion Molecule) is a surface glycoprotein that is highly expressed in epithelial cancers. EpCAM has also been shown to play a morphoregulatory role in normal epithelia and stem/progenitor cells, as well active role in tumor progression in cancer cells.

The transcription factors, according to embodiments herein, include transcription factors affecting the epithelial to mesenchymal transition. It refers to EMT related transcription factors. In an embodiment, the transcription factors include at least one factor selected from TWIST, SLUG, SNAIL and ZEB. TWIST or Twist-related protein 1 (TWIST1) or class A basic helix-loop-helix protein 38 (bHLHa38) is a basic helix-loop-helix transcription factor encoded by the TWIST1 gene. Slug (SNAI2) and Snail (SNAI1) are zinc-finger master regulatory transcription factors for organogenesis and wound healing. The transcription factor family of ZEB (Zinc-finger E-box binding homeobox) includes Zeb1 (zinc finger E-box-binding homeobox 1) and Zeb2. These are known to have crucial roles in EMT and are also essential during normal embryonic development. In an embodiment, the transcription factor is SNAIL.

The term “Plasticity Ratio” or “PR”, as used herein, refers to a ratio of markers associated with epithelial to mesenchymal transitions or mesenchymal to epithelial transition. Percentages of epithelial and mesenchymal markers are calculated to generate the plasticity ratio. It refers to the ratio of total percentage of mesenchymal markers to the total percentage of epithelial markers. According to embodiments herein, PR values are used in characterizing cells invasiveness & growth properties, (PR∝Invasiveness). In an embodiment, the mesenchymal markers that are used in generating PR values include Vimentin and N cadherin. In an embodiment, the epithelial markers that are used in generating PR values include E-cadherin and EpCam. In an embodiment, a PR ratio of <0.7 indicates epithelial characteristics of the cells and a ratio >1 indicates mesenchymal properties of the cells. An increase in this ratio suggests increasing invasiveness of the tumor cell. Embodiments herein include recombinant cancer cells having a modified plasticity ratio as compared to wild-type cancer cells.

### Method

The embodiments disclosed herein provide a method for producing an animal model of spontaneous metastasis. In an embodiment, the method comprises providing a recombinant cell line having a modified plasticity ratio; and implanting the cell line in one or more orthotopic positions in an animal to obtain a model for metastasis. In an embodiment, the method comprises providing recombinant cancer cells having increased plasticity ratio as compared to wild type cancer cells and implanting, orthotopically, said recombinant cancer cells into an immunodeficient mouse. In an embodiment, the plasticity ratio of said recombinant cell is in the range of 0.7 to 1.2. In an embodiment, the recombinant cancer cells are obtained by transfecting wild type cancer cells with a gene construct encoding at least one transcription factor, and a bioluminescence gene to obtain transfectants capable of expressing the transcription factor. In an embodiment, the bioluminescence gene is luciferase gene.

### Gene Construct

The embodiments herein include providing recombinant gene constructs for preparing recombinant cancer cells. The gene construct, according to embodiments herein, includes transcription factor also referred to herein as Gene of interest or GOI, and a constitutive or inducible promoter. The gene may be under the control of constitutive or inducible promoters. In an embodiment, the gene is under the control of a constitutive promoter sequence. In another embodiment, the gene is under the control of an inducible promoter sequence.

The term “gene construct” as used herein, refers to engineered DNA molecules comprising a nucleotide sequence capable of encoding gene product e.g.: proteins and peptides. In an embodiment, it refers to genetic material comprising transcription factor gene. In an embodiment, it refers to gene of transcription factor associated with up and/or down regulation of EMT and/or MET. EMT inducing transcription factors include, but are not limited to, TWIST, SLUG, SNAIL and ZEB.

In an embodiment, gene construct includes gene sequence capable of encoding at least one factor selected from TWIST, SLUG, SNAIL and ZEB1. The gene may be present in one or more copies. In an embodiment, the gene construct may comprise of complete coding DNA sequence of SNAIL. (SNAI1) gene (Gene ID 6615; SEQ ID NO: 1). In another embodiment, the gene construct may comprise of complete coding DNA sequence of TWIST1 gene (Gene ID 7291; SEQ ID NO: 2). In yet another embodiment, the gene construct may comprise of partial coding DNA sequence of SLUG (SNA12) gene (Gene ID 6591; SEQ ID NO: 3). In another embodiment, the gene construct may comprise of complete coding DNA sequence of ZEB gene (Gene ID 6935; SEQ ID NO: 4). In an embodiment, the gene construct comprises SNAIL, gene. It is understood that various minor modification to the gene construct and methods may be apparent to a person skilled in the art without departing from the spirit and scope of the invention disclosed in various embodiments herein. All such modifications are understood to be included within the scope of the appended claims herein. Table 1 provides a list of genes that may be used for achieving the gene construct, according to embodiments herein.

TABLE 1
List of GOIs and Gene IDs.

| | Gene | | | Sequence |
|---|---|---|---|---|
| Sr. | Name; | | | Number |
| no | Source | Type | Gene ID; Description | (IDs) |
| 1. | SNAI1; | Nucleotide | 6615; | Sequence |
| | Homo | sequence | (AF125377.1 Homo sapiens | Number |
| | sapiens | | zinc finger protein (SNAH) | (ID): 1 |
| | (human) | | mRNA, complete cds) | |
| 2. | TWIST1; | Nucleotide | 7291; | Sequence |
| | Homo | sequence | (U80998.1 Human basic helix- | Number |
| | sapiens | | loop-helix DNA binding | (ID): 2 |
| | (human) | | protein (TWIST) gene, | |
| | | | complete cds) | |
| 3. | SLUG | Nucleotide | 6591; | Sequence |
| | (SNAI2); | sequence | (U97060.1 Homo sapiens Slug | Number |
| | Homo | | mRNA, partial cds) | (ID): 3 |
| | sapiens | | | |
| | (human) | | | |
| 4. | ZEB1; | Nucleotide | 6935; | Sequence |
| | Homo | sequence | (AK091478.1 Homo sapiens | Number |
| | sapiens | | cDNA FLJ34159 fis, clone | (ID): 4 |
| | (human) | | FCBBF3013623, highly | |
| | | | similar to NIL-2-A ZINC | |
| | | | FINGER PROTEIN) | |

The gene construct produced may further be cloned into a suitable vector by methods generally known in the art. Suitable vector may be constructed using methods generally known in the art. Suitable vector is preferably one which allows integration of construct in a host cell. Suitable vectors, according to embodiments herein, include plasmid or viral based vectors which may be used for introducing genes via genomic integration in a suitable cancer cell line.

### Vectors

The embodiments herein include providing suitable vectors comprising the gene construct. The vector is an expression vector, e.g.: plasmid or viral, comprising gene construct and modifications as disclosed in various embodiments herein. Suitable cancer cell lines may be transfected using plasmid vector under the regulation of constitutive or inducible promoters. In an exemplary embodiment, the vector is a constitutive vector e.g., Vector 1026. Vector 1026, according to embodiments herein, comprises the gene construct along with a bioluminescence gene capable of expression of fluorescent marker protein. In an embodiment, the fluorescent marker protein is EmGFP (Emerald Green Fluorescent Protein). Any suitable fluorescent marker protein may be used such as, but not limited to, YFP (yellow fluorescent protein), BFP (blue fluorescent protein), CFP (cyan fluorescent protein), Orange FP, Red FP, Emerald, Superfolder GFP, Azami Green, etc. In another exemplary embodiment, the vector is an inducible vector, e.g., Vector 1027. Vector 1027, according to embodiments herein, comprises the gene construct along with a tetracycline promoter sequence (TRE promoter). Tetracycline promoter is an inducible promoter.

The plasmid vectors, according to embodiments herein, also includes a selectable antibiotic marker (antibiotic resistance gene). In an embodiment, the vector includes one or more antibiotic resistance gene cassettes. Examples of antibiotic resistance genes includes, but is not limited to, neo (neomycinphosphotransferase), hyg (hygromycin resistance gene), zeo (zeocin resistant Sh ble gene), bla (blasticidin resistant bsd gene), and puro (puromycin N-acetyl-transferase). In an embodiment, the antibiotic resistance gene comprises zeo gene (Gene ID: 837731; Source: Arabidopsis thaliana) cassette. In another embodiment, the antibiotic resistance gene comprises bla gene (Gene ID: 41989405; Source: Lachnellula hyaline) cassette.

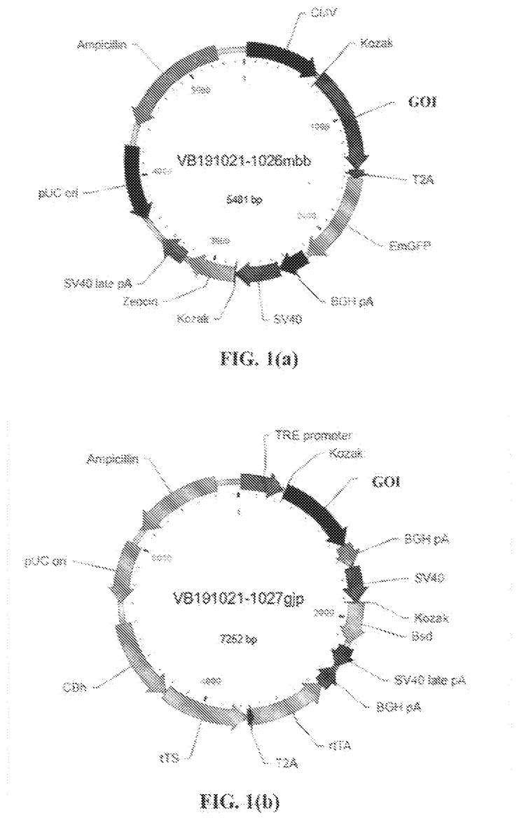

FIG. 1 is a schematic representation of vectors containing gene of interest (GOI) and selectable marker used in the study, wherein 1(a) depicts a constitutive vector; and 1(b) depicts an inducible vector, according to embodiments disclosed herein.

### Recombinant Cancer Cells

The embodiments herein include providing recombinant cancer cells produced by genetically modifying cancer cells. Cancer cells that may be modified include wild type cancer cells such as primary tumor cells and/or cancer cell lines established therefrom. According to embodiments herein, the method includes genetically modifying epithelial carcinoma cell lines.

Various established epithelial carcinoma cell lines originating from different sites are commercially available and may be used in various embodiments herein. In an embodiment, colorectal cancer cell lines are used. In an embodiment, the recombinant cancer cells are obtained by genetically modifying colorectal cancer cell lines. Examples of colorectal cancer cell lines include, but is not limited to, HT29 and SW480. In an exemplary embodiment, HT29, a human colorectal adenocarcinoma cell line, is used. In another exemplary embodiment, SW480, a primary adenocarcinoma cell line, is used.

The recombinant cells, according to embodiments herein, can express transcription factors which further regulate the expression of EMT markers. The recombinant cells are screened to isolate stable cells having an increased plasticity ratio. The recombinant cell lines, according to embodiments herein, include gene construct for transcription factor, also referred to herein as Gene of interest or GOI. The gene may be under the control of constitutive or inducible promoters. In an embodiment, the gene is under the control of a constitutive promoter sequence. In another embodiment, the gene is under the control of an inducible promoter sequence.

In an embodiment, the method for producing recombinant cancer cells comprises transfecting wild type cells with a gene construct encoding transcription factor, and a luciferase gene, to obtain transfectants capable of expressing the transcription factor. In another embodiment, the method for producing recombinant cancer cells comprises transfecting wild type cells with a gene construct encoding transcription factor; screening and selection of stable transfectants based on PR values; and transfecting stable clones with a luciferase gene to obtain transfectants capable of expressing the transcription factor and luciferase.

In an embodiment, the method for producing recombinant cancer cells comprises transfecting wild type cells with a gene construct encoding SNAIL, and a luciferase gene, to obtain transfectants capable of expressing SNAIL. In another embodiment, the method for producing recombinant cancer cells comprises transfecting wild type cells with a gene construct encoding SNAIL; screening and selection of stable transfectants based on PR values; and transfecting stable clones with a luciferase gene to obtain transfectants capable of expressing the transcription factor and luciferase.

In another embodiment, the method for producing recombinant cancer cells comprises transfecting wild type cells with a gene construct comprising transcription factor; selecting stable transfectants; propagation and characterization of clones; screening for suitable clones based on PR, transfecting stable clones with a luciferase gene to obtain transfectants capable of expressing the transcription factor and luciferase.

In another embodiment, the method for producing recombinant cancer cells comprises transfecting wild type cells with a gene construct comprising SNAIL; selecting stable transfectants; propagation and characterization of clones; screening for suitable clones based on PR, transfecting stable clones with a luciferase gene to obtain transfectants capable of expressing the SNAIL and luciferase.

The recombinant cells are screened to isolate stable cells having increased plasticity ratio. Generally known strategies and methods of transfection, selection and propagation may be used in various embodiments.

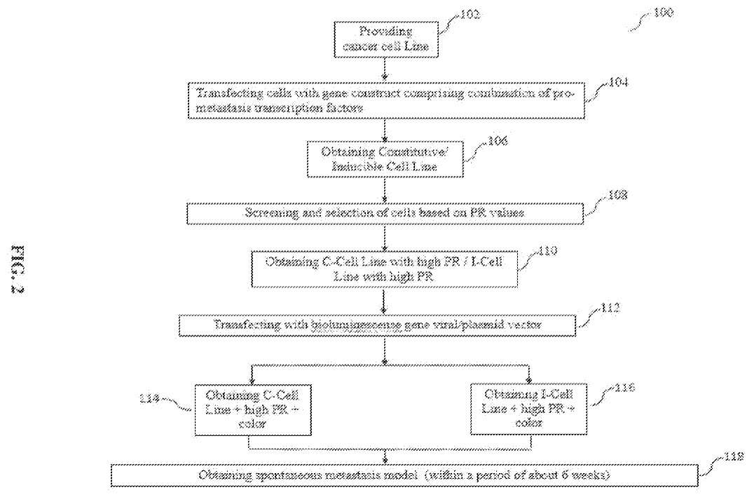

FIG. 2 is a flowchart illustrating different strategies for constructing cells with high Plasticity Ratio (PR) and producing the model, according to embodiments disclosed herein. The method (100) comprises providing cancer cells (102), wild type cancer cells or established cancer cell lines; transfecting (104) cells with a gene construct comprising pro-metastasis transcription factor that is capable of regulating markers associated with EMT. The gene construct may be inducibly and/or constitutively regulated and therefore, may include suitable inducible and/or constitutive promoter sequences. The transfected cells are further screened to select stable transfectants, using antibiotic selection (not shown). The method further comprises obtaining (106) stable transfectants capable of constitutive and inducible expression of the transcription factors. The stable transfectants may then be propagated and characterized as per suitable requirement. The method (100) further includes screening and selection (108) of suitable cells based on PR values; and obtaining constitutive/inducible cell line (110) having high PR and/or inducible cell line capable of having high PR. The selection of cells based on PR values may be performed using any method generally known to assess plasticity of cells. In an embodiment, PR ratio may be calculated by measuring cell characteristics using generally known methods, e.g.: flow cytometry and immunofluorescence. In an embodiment, the screening and selection (108) of cells based on PR values to obtain (110) cells having high PR values is performed using a system and method as described in PCT/IN2021/050915. Contents of PCT/IN2021/050915 are herein incorporated by reference in its entirety. The method (100) further comprises transfecting (112) the constitutive/inducible clones with bioluminescence gene and obtaining (114 and 116) transfectants having high PR and capable of expressing the bioluminescence gene. Transfection for introduction of the bioluminescence gene may be via plasmid or viral mediated transfection. The method (100) achieves constitutive cell lines (114) having high PR value and capable of bioluminescence; and inducible cell lines (116) having high PR value and capable of bioluminescence. The method (100) includes implanting the recombinant cells into one or more orthotopic locations in mouse to obtain (118) the spontaneous metastasis model.

### Transfection

Transfection may be carried out by methods generally known in the art. In an embodiment, the method for stable transfection comprises of plating healthy and actively growing epithelial cancer cells to obtain confluent growth; transfecting the cells with plasmid DNA, preferably on day 2; and selecting antibiotic-resistant cells, preferably from day 3 or 4 onwards, followed by screening and identification of clonal populations. The monoclones may be isolated using different techniques such as, but not limited to, limiting dilution, cloning rings and trypsin discs, Fluorescence Activated Cell Sorting (FACS), and automated clone picking. In an embodiment, the clones are isolated by limiting dilution and expansion technique.

The method, according to embodiments herein, further includes, selecting stable transfectants by antibiotic selection. Examples of selectable antibiotic markers include, but are not limited to, G418, hygromycin B, zeocin, blasticidin and puromycin. In an embodiment, the selectable antibiotic marker for constitutive plasmid is Zeocin. In another embodiment, the selectable antibiotic marker for inducible plasmid is blasticidin.

Embodiments herein includes screening and selection of stable transfectants. Any suitable method may be used for screening and selection of stable transfectants. Use of antibiotic resistance gene cassettes is generally used in screening of stable transfectants which may also be employed in various embodiments herein.

### Screening of Clones

The suitable genetically modified clones are selected based on plasticity ratio. Characterization of clones is performed by techniques such as PCR for analyzing clone ability to carry the GOI; western blot to show protein overexpression (from the genes of interest); and flow cytometry to check the change in expression levels of protein of interest, e.g., Vimentin, E-Cadherin, N Cadherin, EpCAM, CD 133, CD 44, Snail, etc. In an embodiment, the screening is by performing a multitude of functional assays as disclosed in PCT/IN2021/050915, incorporated herein by reference in its entirety.

In an embodiment, the method comprises evaluating selected clones using screening platform described in PCT/IN2021/050915 (also referred as METAssay platform).

In an embodiment, the screening method includes: characterizing cells by evaluating chemosensitivity by: determining epithelial to mesenchymal ratio in a tumor by evaluating parameters such as determining plasticity ratio, stemness of the cell, and doubling time of tumor; evaluating the ability of the cells to move out of epithelial layer by evaluating parameters such as, adhesion, migration, and invasion; evaluating the ability of the cell to enter the endothelial system (blood), survive in the endothelial system and migrate from the endothelial system to a secondary site by evaluating parameters such as intravasation, Tumor Cell Induced Platelet Aggregation (TCIPA), and extravasation; evaluating the ability of the cells to survive in the secondary site, cross talk with the tissue in the secondary site and successfully grow back to a tumor in the secondary site by evaluating parameters such as, Mesenchymal to Epithelial transition (MET), apoptosis, metabolism, and Exosome vesicles secretion and uptake; and determining the ability of tumor cells to survive in a hostile environment by performing extrinsic assays evaluating parameters such as, cytotoxicity, angiogenesis, immune profiling, autophagy analysis, effect of hypoxia, and cell cycle analysis by Ki67. Generally known techniques and assay kits may be used in evaluating chemosensitivity; parameters such as, adhesion, migration, and invasion, intravasation, Tumor Cell Induced Platelet Aggregation (TCIPA), extravasation, Mesenchymal to Epithelial transition (MET), apoptosis, metabolism, Exosome vesicles secretion uptake, cytotoxicity, angiogenesis, immune profiling, autophagy analysis, effect of hypoxia, and cell cycle analysis by Ki67.

The recombinant cells or clones, according to embodiments herein, having a changed phenotype of less tumorigenicity but more invasiveness i.e., high PR are selected for further steps.

### Transduction

The method according to embodiments herein, further includes transducing or transfecting clones having higher PR, as compared to wild type cancer cells, with a plasmid vector or a lentiviral vector comprising one or more reporter genes. In an embodiment, the reporter gene is a bioluminescence gene. Examples of common bioluminescence gene include, but are not limited to, fluorescence proteins (such as GFP and RFP), enzymes such as beta-galactosidase and luciferase. Luciferase expression system is preferably used in various embodiments herein. In an embodiment, transfection/transduction is carried out with a plasmid or a lentiviral vector comprising a luciferase gene.

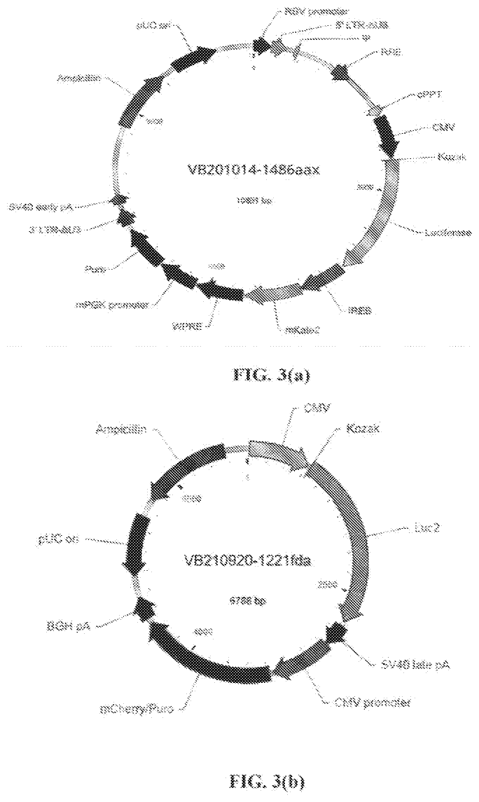

Accordingly, in an embodiment, the transduction includes using lentiviral particles (VB2014-1486aax) expressing firefly luciferase gene under CMV promoter along with fluorescence and antibiotic resistance gene. In an embodiment, the fluorescence gene is mKate2 (derived from mKate by mutation; M1_S2insV/V45A/M146T/S158A/K231R; Source Entacmaea quadricolor). Further, in an embodiment the lentiviral based vector includes puromycin n-acetyl-transferase (pac; Gene ID: 54318529; Source Aspergillus lentulus) as antibiotic resistance gene. FIG. 3 is a schematic representation of luciferase encoding vectors, wherein 3(a) depicts a lentiviral vector (VB2014-1486aax); and 3(b) depicts a plasmid vector (VB210920), according to embodiments disclosed herein.

In an embodiment, the transduction of cells with luciferase lentiviral particle is achieved by inoculating cells, preferably in a 6 well plate, to achieve desired confluency. In an embodiment, the desired confluency is about 30 to 50% in about 18 to 24 hours. Further, in an embodiment, after incubation, the cells may be infected with viral particles (based on multiplicity of infection) in a fresh medium in minimal volume for 8 to 12 hours. For enhancing efficiency of lentiviral infection, the media may contain polybrene at predetermined concentration. Additionally, cells expressing luciferase may be selected based on antibiotic selection. In an embodiment, luciferase expression may be confirmed by in vitro bioluminescence production assay in the presence of luciferin substrate.

In another embodiment, the transfection of cells may be carried out using luciferase encoded plasmid vector. In an exemplary embodiment, vector 10920 may be used. Vector 10920, according to embodiments herein, includes luciferase gene (Luc2; Gene ID: 116160065; Source: Photinus pyralis): gene for fluorescent marker protein (mCherry; GenBank: AY678264.1; Source synthetic construct) and an antibiotic resistance gene (puromycin n-acetyl-transferase (pac)). Further in an embodiment, antibiotic selection and single cell dilution may be carried out for screening and isolating clones.

In an embodiment, the clones having higher PR and capable of luciferase expression are selected for animal model studies. Various methods of producing gene constructs and recombinant host cells are generally known to a person skilled in the art which may be practiced in achieving the embodiments herein.

### Implantation of Recombinant Cells in a Suitable Animal Model

The animal model, according to embodiments herein, is a non-human immunodeficient animal. In a preferred embodiment, the immunodeficient animal model is a NOD-SCID mice. The animal model, according to embodiments disclosed herein, is characterized in that the model can form a tumor and metastasizing at a faster rate, post the orthotopic implanting of the recombinant cell line into the animal. Implantation may be performed by generally known surgical methods. In an embodiment, the recombinant cells having higher PR are implanted by surgery in one or more orthotopic positions in NOD-SCID mouse. In an embodiment, the model is an immunodeficient mouse comprising orthotopic implant of recombinant cancer cells having modified plasticity ratio as compared to wild type cancer cells, wherein said recombinant cancer cells are obtained by transfecting wild type cancer cells with at least two transcription factor gene, and bioluminescence gene.

The model, according to embodiments herein, can exhibit early metastasis. In one embodiment, metastasis is observed within 1 to 6 weeks of implantation. In an embodiment, the suitable time is at least a period in the range of 1 to 6 weeks. In an embodiment having model with constitutive recombinant cells, metastasis may be observed in a period as early as 1 week after implantation into suitable mouse. The model after implantation may, therefore, be allowed a period of about 5 to 7 days for growth of primary tumor. The recombinant cancer cells having increased PR values, are capable of exhibiting early metastasis. In an embodiment, the recombinant cells are allowed a suitable time period, preferably at about 1 to 6 weeks, to metastasize. Early metastasis may further be facilitated by various other pro-metastatic factors. Such factors, for e.g.: diet, environmental factors, etc., which accelerate metastasis may be apparent to a person skilled in the art and used in various embodiments herein.

In an embodiment, the models comprising inducible recombinant cells are subjected to tetracycline or doxycycline-rich diet. Vector 1027, according to embodiments herein, comprises the gene construct along with a tetracycline promoter sequence (TRE promoter). A tetracycline or doxycycline rich diet provided facilitates stringent expression protocols. The expression patterns may be modulated through diet to facilitate switching between high PR value and low PR value patterns, to suit requirement. In an embodiment, the method includes administering tetracycline or doxycycline-based diet, to the mouse, to obtain increase in PR value. The scheme of diet may be varied as per requirement. In an embodiment, the diet scheme comprises, administering a tetracycline or doxycycline diet for a time period ranging from 0 to 40^{th} day, post implanting of recombinant cells into the mouse.

In another embodiment, the diet scheme comprises, administering normal chow for a time period ranging from 0 to 14^{th} day; and tetracycline or doxycycline diet for a time period ranging from 15^{th} to 40^{th} day, post implanting of recombinant cells into the mouse.

In yet another embodiment, the diet scheme comprises, administering normal chow for a time period ranging from 0 to 7^{th} day; tetracycline or doxycycline diet for a time period ranging from 8^{th} to 21^{st}; and again, normal chow for a time period ranging from 22 to 40^{th} day, post implanting of recombinant cells into the mouse.

Embodiments herein enable visual imaging of metastasis progression by non-invasive techniques. Non-invasive imaging techniques generally include bioluminescence (optical), positron emission tomography (PET)/X-ray computed tomography (CT), and magnetic resonance imaging (MRI). Embodiments herein are capable of Luciferase expression-assisted bioluminescence imaging. Such techniques can generally provide complementary information and accurately measure tumor growth which can be correlated with histopathological analysis. Imaging of the location and volume of primary and metastatic foci can be achieved by recording luminescence images of the animal's whole body after the administration of luciferin.

In an embodiment, for performing kinetic study or evaluating metastasis, the animal is injected with luciferin substrate via intraperitoneal route before imaging. Luciferin substrate may be administered at any suitable amounts over a suitable period, as may be known to a person skilled in the art. In an exemplary embodiment, a 20 g mouse is intraperitoneally injected with 100 μl luciferin substrate before imaging. Further in an embodiment, imaging is carried out after a period of 10 minutes. Thus, luciferase expressing cells producing bioluminescence enable evaluation by live imaging.

### Uses

Embodiments herein achieve a biologically relevant orthotopic spontaneous metastasis model that is robust, statistically significant, cost-effective, and time sensitive. The model, according to embodiments disclosed herein, may be used for screening of pharmaceutical substances, such as NCEs (New Chemical Entity's) and other potential therapeutic candidates including chemical drugs, biotherapeutics, biosimilars, etc. Further, the embodiments herein may also be used in evaluating drugs for repurposing, for their potential use in inhibiting cancer metastasis. The method, according to embodiments herein, includes kinetic tracking of cells in vivo, using luciferase-based bioluminescence signal capable of being imaged by a suitable instrument. The method also includes use of constitutive and/or inducible vector capable of improving stringency of the model. The model, according to embodiments disclosed herein, may be used for colorectal cancer liver metastasis.

In an embodiment, the method for screening substance for anti-metastatic or anti-cancer activity, comprises administering the substance to the model; allowing the cancer cell lines to metastasize for a suitable time period; and determining anti-metastatic or anti-cancer activity by analogy with control or control model. Control or control models may include positive or negative controls including, but not limited to, animal, immunodeficient or non-immunodeficient; treated or untreated; with or without primary tumor e.g.: colorectal cancer; and with or without metastasis e.g.: liver metastasis. Controls may further include any reference data or live models e.g.: in vitro models, in vivo models etc., or data obtained therefrom, which may be used to assess and evaluate metastasis, anti-metastatic activity, anti-cancer activity, etc. In an embodiment, control includes a nude or NOD-SCID mouse. The anti-metastatic or anti-cancer activity is assessed by measuring the inhibitory or preventive effect of said substance on metastasis. Measuring the inhibitory or preventive effect may be by visual imaging or measurement of intensity of bioluminescence. Ways of assessment of anti-metastatic or anti-cancer activity, and measurement of inhibitory or preventive effect of substances and therapy would be known to one skilled in the art. In an embodiment, the anti-metastatic or anti-cancer activity is determined by luciferase assisted visual imaging.

Embodiments herein include a method for evaluating metastasis. In an embodiment, the method comprises implanting the recombinant cell line in one or more orthotopic positions in an animal; administering said animal with luciferin; and monitoring metastasis in said animal by suitable visual imaging. Implantation may be performed by generally known surgical methods. Evaluation of metastasis may be performed at period as early as 4 to 6 weeks post implantation.

Embodiments herein include induction of high PR value in the method for evaluating metastasis. In an embodiment, the method comprises implanting the recombinant cell line in one or more orthotopic positions in an animal; administering the animal with tetracycline or doxycycline rich diet by following specific diet scheme; and monitoring metastasis in the animal by suitable visual imaging.

Embodiments herein also include a method for screening anticancer and/or antimetastatic drug candidates. In an embodiment, the method comprises providing the orthotopic animal model; administering the drug candidate to the animal model; and evaluating drug candidate's effect on metastasis in the animal by luciferase assisted visual imaging. The animal is injected with luciferin before imaging to enable kinetic bioluminescence imaging. In an embodiment, the method comprises providing the orthotopic animal model; administering a luciferin injection for facilitating bioluminescence imaging; identifying level of metastasis in said animal; administering the drug candidate to the animal model; and evaluating metastatic response to the drug candidate in the animal. The evaluation may be performed to understand if the drug candidate reduces and/or delays metastasis in said animal as compared to the untreated level in a control. The drug candidate capable of increased reduction or delay in metastasis may be selected as the most suitable candidate based on the metastatic response to one or more drug candidate. Anticancer drug candidates, according to embodiments herein, include any drug candidate suitable for treating or managing cancer and/or metastasis including, but not limited to, NCEs, repurposed drugs, antineoplastic agents, etc. In an embodiment, the drug is MS-AP_003. Negative control drug candidates, according to embodiments herein, include any drug candidate that is not suitable for treating or managing cancer and/or metastasis. In an embodiment, the drug is MS-AP 006.

Embodiments are further described herein by reference to the following examples by way of illustration only and should not be construed to limit the scope of the claims provided herewith.

### Example 1: Transfection Protocol

At Day 0, healthy and actively growing epithelial carcinoma cells were seeded in each well of a 6-well plate. After obtaining a 70 to 90% confluency transfection was carried out. About 1 h before performing transfection, the culture medium was aspirated from the wells. 1.75 mL pre-warmed Opti-MEM™ Reduced Serum Medium was added to the well with cells and plasmids with GOI, while 2 mL of medium was added to “No transfection, no Lipofectamine” control well respectively. The cells were kept at 37 degrees Celsius for incubation till DNA-Lipofectamine® 3000 complex was ready for transfection. 3.75 μL [2:3 (m/v) DNA to Lipofectamine] and 7.5 μL [1:3 DNA (m/v) to Lipofectamine] Lipofectamine® 3000 Reagent was added to 121.25 μL and 117.5 μL OptiMEM™ respectively in two separate 1.5 mL tubes followed by shaking. DNA-P3000 master mix was prepared by diluting 2 μg of DNA (gene of interest or control plasmid) in Opti-MEM™ in duplicate followed by addition of 4 μL of P3000 reagent to each mixture. The volume of the added Opti-MEM™ was adjusted such that the total volume of each mixture was 125 μL. The cells were mixed well by pipetting up and down. 125 μL of DNA-P3000 mixture was then added to 125 μL of Lipofectamine mixture followed by mixing. 125 μL of OptiMEM™ media was also added to Lipofectamine mixture for Lipofectamine control well. The tubes were then incubated at room temperature for 20 to 30 min. 250 μL of DNA-Lipofectamine mixture or Lipofectamine only (Lipofectamine control) was then added to each well of the 6-well plate (dropwise) while swirling the plate gently to facilitate even distribution of the DNA-Lipofectamine mixture. The cells were then incubated at 37 degrees Celsius for 4 to 6 h. The media was then gently aspirated, and 2 mL fresh complete media was added followed by incubation at 37 degrees Celsius. On day 2, cells were observed under an inverted fluorescence microscope. From day 3 onwards, the spent media was removed and fresh media containing selection antibiotic (Zeocin for constitutive transfection and Blasticidin for inducible transfection) was added according to fixed dose for each cell line determined by kill curve experiments. The transfected cells were maintained in complete growth medium containing selection antibiotic till untransfected cells died and transfected cells started forming colonies/grew into a confluent culture/developed isolated colonies. From day 8 onwards, depending on the growth characteristics of the transfected cells, Clonal Selection or Colony Selection method was carried out. Post clonal selection, the cells containing inducible vector were maintained with 1 μg/mL or 3 μg/mL of Tetracycline for at least 5 days and analysis of the gene of interest was performed.

### Example 2: Characterization of Clones Expressing Luciferase

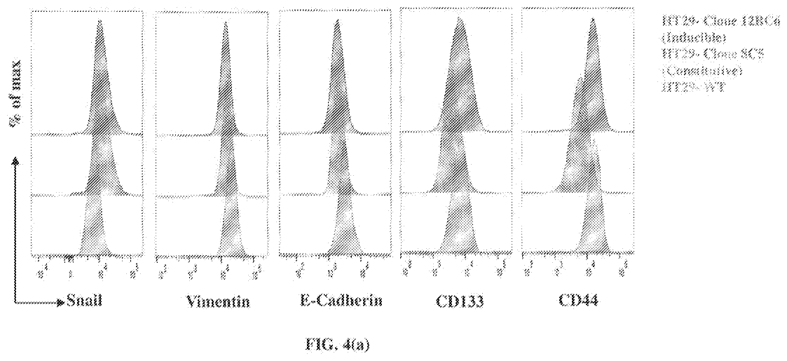

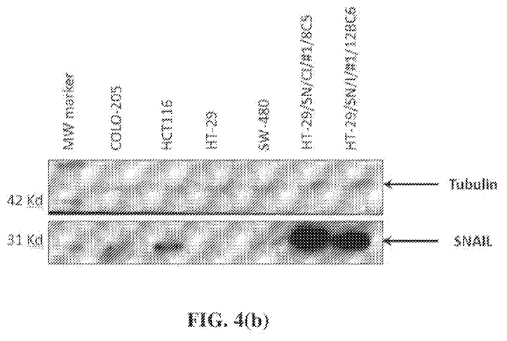

Luciferase expressing clones were confirmed for retention of their high PR ratio by assessing expression of various markers by flow cytometry. FIG. 4(a) is a graph depicting flow cytometry analysis of HT29 clone 12BC6 (inducible; clone 1) and HT29 clone 8C5 (constitutive; clone 2) for expression of Snail, Vimentin, E-Cadherin, CD 133, and CD 44 proteins. An increase in plasticity ratio in luciferase expressing clones was confirmed by decrease in E-cadherin and increase in stem cell marker CD133. FIG. 4(b) is a western blot gel image depicting protein expression for Tubulin (loading control) and SNAIL protein in COLO-205, HCT-116, HT-29, SW-480, HT29 clone 8C5 and HT29 clone 12BC6 cell lines. An amplified expression of SNAIL protein was confirmed for HT29 clone 8C5 and HT29 clone 12BC6.

### Example 3: Decrease in Tumorigenesis Due to High PR

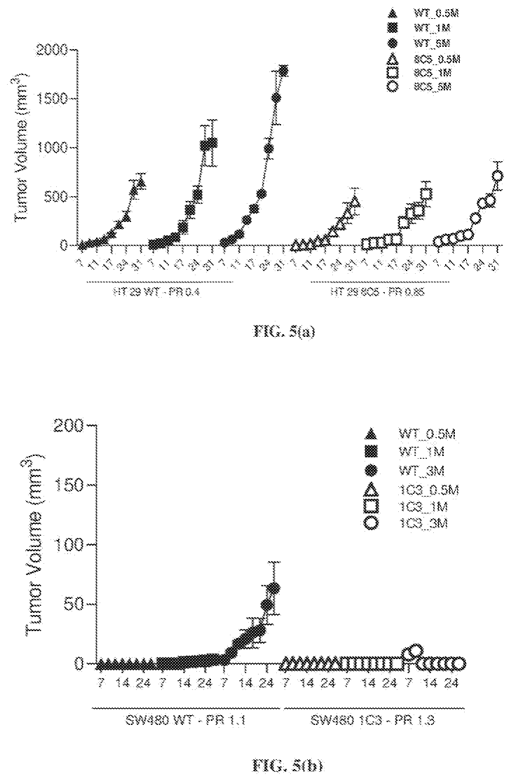

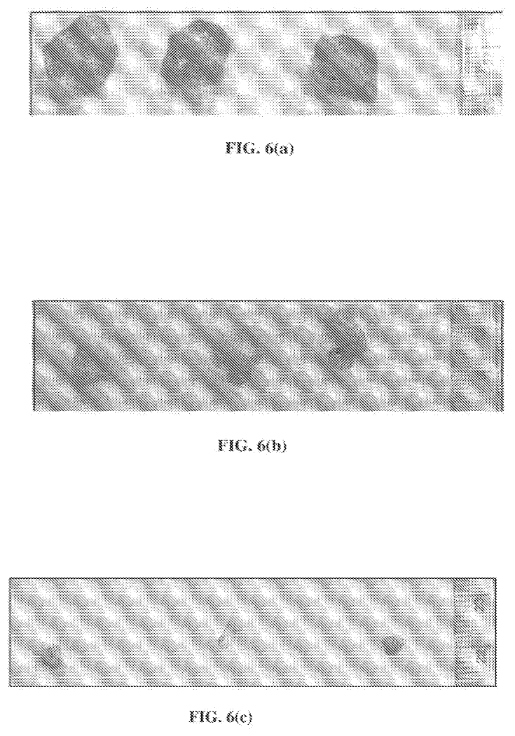

According to the present heterotopic tumor implantation model in NOD-SCID mice, high PR cell lines present significantly slower primary tumor growth as compared to low PR cell lines. Low PR and high PR clones of cell line HT29 and SW480 were transplanted subcutaneously in right flank of NOD-SCID mice in three different cell numbers, 0.5, 1, & 5 million/mouse. Tumors were measured with digital Vernier calipers and volume was recorded as [(length×width×width)/2] (mm³). FIG. 5 are graphical representations depicting tumor volume at different number of cells transplanted in NOD-SCID mice (0.5, 1, & 5 million/mouse respectively), wherein 5(a) illustrates a comparison of tumor volume in HT29 WT cells (with PR ratio of 0.4) and HT29 8C5 cells (constitutive clone of HT29 with PR ratio of 0.85); while 5(b) illustrates a comparison of tumor size in SW480 WT cells (with PR ratio of 1.1) and SW480 1C3 (constitutive clone of SW480 with PR ratio of 1.3). As can be seen from the graph, HT29 8C5 and SW480 1C3 cells having high PR value (0.85 and 1.3 respectively) showed low tumor volume as compared to HT29 WT and SW480 WT cells with lower PR value (0.4 and 1.1 respectively). FIG. 6 are pictorial images depicting tumor size; wherein left, center and right images correspond to number of cells transplanted in NOD-SCID mice (0.5, 1, & 5 million/mouse respectively); 6(a) illustrates tumor size in NOD-SCID mice injected with HT29 WT cells; 6(b) illustrates tumor size in NOD-SCID mice injected with HT29 8C5 cells; and 6(c) illustrates tumor size in NOD-SCID mice injected with SW480 WT cells.

### Example 4: Effect on Tumorgenicity by Luciferase Transduced Cells

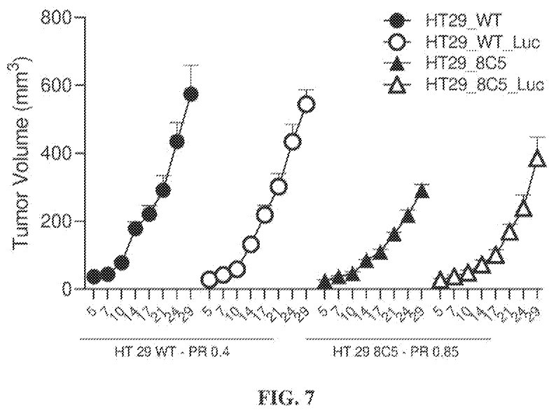

The effect of luciferase transduction on tumorigenicity of clones was further analyzed. Animals were injected with luciferase expressing and non-expressing clones (HT29_WT, HT29_WT_Luc, HT29_8C5, HT29_8C5_Luc) and tumor volumes were measured. FIG. 7 is a graph depicting effect of luciferase on tumor volume in NOD-SCID mice injected with different cells: HT29_WT, HT29_WT_Luc, HT29 8C5, HT29 8C5_Luc, respectively. No significant difference in tumorigenicity was seen in clones expressing luciferase as compared to cells without luciferase. Moreover, the HT29_8C5_Luc clones having higher PR (0.85) showed less tumorigenicity than the HT29_WT_Luc clones having lower PR (0.4), similar to the data shown in FIG. 5.

### Example 5: Tetracycline (tet) Induction

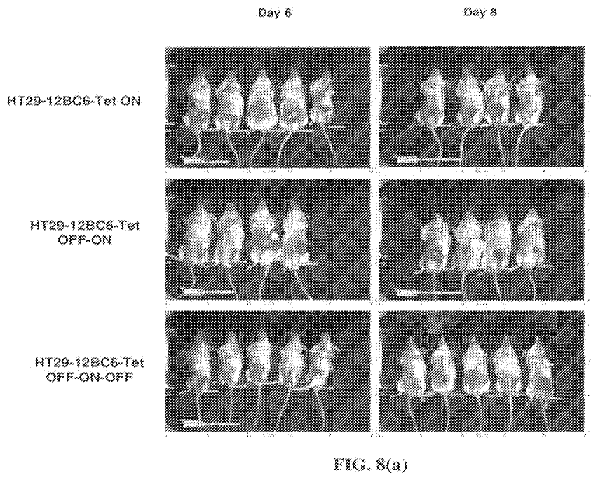

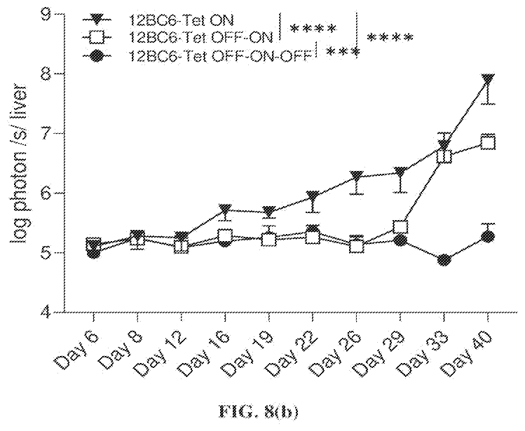

The metastatic potential of high PR inducible clone of colorectal cancer cell line in orthotopic model of colon carcinoma was analyzed. Briefly, animals were anesthetized using xylazine and ketamine. The ceco-colic junction of cecum was exposed after laparotomy and 100,000 cells in 20 μL McCoy's media+Matrigel (1:1) were carefully injected in the cecal wall ensuring that injection was not deposited in cecal lumen. The incision was closed in layers and animals were monitored till recovery from anesthesia. Animals were provided either normal chow or Doxycycline (a long-lasting isomer of tetracycline) diet (625 mg/kg) for tetracycline mediated induction of high PR in tumor cells as per the treatment schemes. In some animals, liver and mLN metastasis were observed post 5 days of inoculation. FIG. 8(a) are bioluminescence images depicting NOD-SCID mice injected with HT29-12BC6_Luc cells with different strategies for induction i.e., Tet on, Tet off-on, and Tet off-on-off at day 6 and day 8 respectively, post tumor cell implantation and with differing schemes for tetracycline mediated high PR induction. The different schemes for tetracycline mediated high PR induction include, Tet on—where doxycycline diet was provided throughout the study; Tet off-on—where doxycycline diet was provided 2 weeks after tumor cell transplantation for late induction of high PR; and Tet-off-on-off where normal chow was provided for one week post tumor cell implantation followed by doxycycline diet for two weeks and then normal chow for another week so as to develop a low PR-high PR-low PR profile in the tumor cells. FIG. 8(b) is a graph depicting bioluminescence in livers of NOD-SCID mice injected with HT29-12BC6_Luc Tet on cells, HT29-12BC6_Luc Tet off-on cells and HT29-12BC6_Luc tet-off-on-off cells respectively, as observed till day 40. Overall, when animals were provided Doxycycline diet (broad spectrum tetracycline-class antibiotic) throughout the study period (tet on), they presented highest rate and intensity of metastasis as observed by repeated bioluminescence imaging performed twice a week. Similar observations were made when doxycycline was introduced two weeks after cancer cells implantation, allowing establishment of solid tumors.

### Example 6: Endpoint Study on NOD-SCID Mice

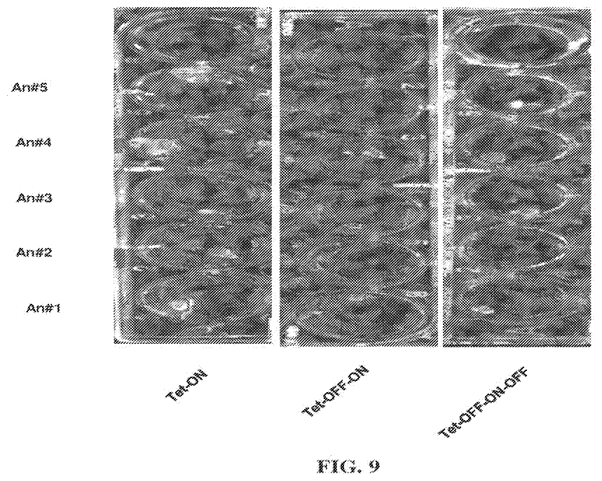

To confirm the findings obtained using inducible luciferase clones, similar study was performed, but endpoint, in NOD-SCID mice. FIG. 9 are bioluminescence images depicting mesenteric lymph nodes (mLN) isolated from NOD-SCID mice injected with HT29-12BC6_Luc Tet on cells, HT29-12BC6_Luc Tet off-on cells and HT29-12BC6 tet-off-on-off cells (An #1, An #2, An #3, An #4, and An #5 represent animal number 1, 2, 3, 4, and 5 respectively). Higher incidences of mLN invasion of tumor cells were observed in HT29-12BC6_Luc Tet on group. The 12BC6_Luc Tet off-on cells, did not show any metastasis till the time tet was not given, highlighting the efficacy of the inducible vector, as metastasis developed upon induction with Tet. Conversely, HT29-12BC6 tet-off-on-off cells did not show any metastasis, as the time given on induction was less.

### Example 7: High PR Leads to Higher Metastasis

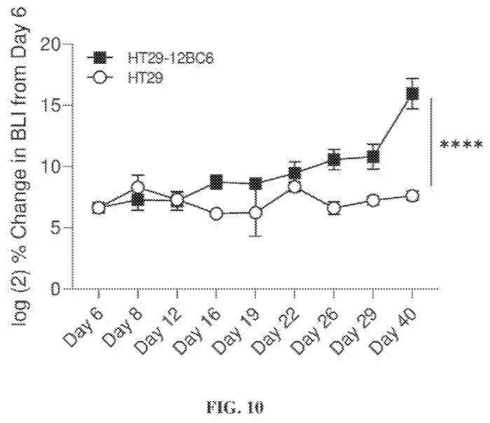

While different Tet ON schemes generate a variation in PR of inducible clones, to establish increased metastasis with High PR cell lines, low PR WT cell lines were compared. FIG. 10 is a graph depicting relative bioluminescence intensity over liver in mice injected with HT29 WT_Luc and HT29-12BC6_Luc cells. As expected, the high PR inducible cell line was significantly more metastatic (measured by bioluminescence intensity over liver) than low PR WT cell line.

### Example 8: Drug Repurposing

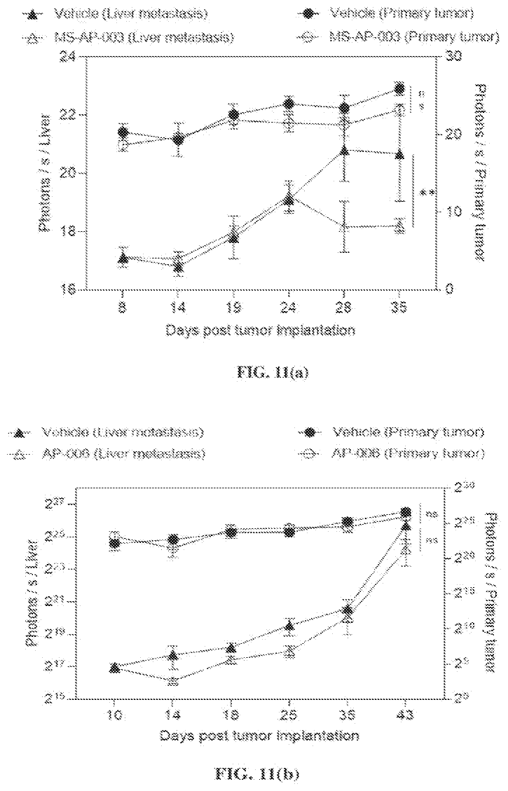

Using in vitro platform, screening was performed to identify NCEs as well as to repurpose already established drugs for anti-metastatic therapy. MS-AP-003 was found to be a good candidate for testing in vivo metastasis animal model, as was identified by the METAssay platform. Luciferase expressing inducible cell line HT29 12BC6 was implanted in cecum of NOD-SCID mice. From day 4 onwards, MS-AP-003 was dosed at 150 mg/kg P.O. every day. In agreement with the in vitro results, MS-AP-003 successfully reduced liver bioluminescence intensity in treated animals indicating reduction in liver metastasis. Conversely, MS-AP-006 did not have any effect in the in vitro METAssay platform and was therefore chosen to dose as a negative control, to showcase the selective and specific nature of the animal model. From day 7 onwards, MS-AP-006 was dosed at 60 mg/kg P.O. for the first day and then a maintenance dose of 2 mg/kg every day. FIG. 11(a) is a graph depicting liver bioluminescence intensity in animals with orthotopic implantation of HT29-12BC6_Luc cells and treated with MS-AP-003. FIG. 11(b) is a graph depicting liver bioluminescence intensity in animals with orthotopic implantation of HT29-12BC6_Luc cells and treated with AP-006.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A method for producing an animal model of metastasis, said method comprising:
providing recombinant cancer cells obtained by transfecting the wild type cancer cells with a vector comprising a gene construct encoding at least one transcription factor selected from a group consisting of TWIST, SLUG, SNAIL and ZEB, and a bioluminescence gene to obtain transfectants capable of expressing said transcription factor; and
implanting, orthotopically, said recombinant cancer cells into an immunodeficient mouse, wherein the plasticity ratio of said recombinant cell is in the range of 0.7 to 1.2.

2. The method as claimed in claim 1, wherein said metastasis is liver metastasis of colorectal cancer.

3. The method as claimed in claim 1, wherein said cancer cells are colorectal cancer cells.

4. The method as claimed in claim 3, wherein said wild type cancer cells are obtained from at least one cell line selected from a group consisting of HT29 and SW480.

5. The method as claimed in claim 1, wherein said gene construct comprises a nucleotide sequence selected from a group consisting of SEQ ID NO: 1, SEQ ID NO: 2, SEQ ID NO: 3 and SEQ ID NO: 4.

6. The method as claimed in claim 5, wherein said gene construct comprises of nucleotide sequence of SEQ ID NO. 1.

7. The method as claimed in claim 1, wherein said transcription factor is SNAIL.

8. The method as claimed in claim 1, wherein said vector comprises a constitutive promoter for constitutive expression of the transcription factors.

9. The method as claimed in claim 1, wherein said vector comprises an inducible promoter capable of regulating expression of the transcription factors.

10. The method as claimed in claim 1, said method further comprising allowing the recombinant cancer cells to metastasize for a suitable time period.

11. The method as claimed in claim 10, wherein said suitable time is at least a period in the range of 1 to 6 weeks.

12. The method as claimed in claim 1, wherein said mesenchymal marker is selected from N-Cadherin and Vimentin.

13. The method as claimed in claim 1, wherein said epithelial marker is selected from E-cadherin and EpCam.

14. The method as claimed in claim 1, wherein said plasticity ratio of said recombinant cancer cells is in the range of 0.8 and 1.2.

15. The recombinant cancer cells as claimed in claim 14, wherein said plasticity ratio is in the range of 0.9 and 1.1.

16. An animal model of metastasis obtained by the method claimed in claim 1.

17. The model as claimed in claim 16, comprising orthotopic implant of recombinant cancer cells having modified plasticity ratio as compared to wild type cancer cells.

18. The model as claimed in claim 16, wherein said mouse is NOD-SCID mouse.

19. A method for screening a substance for anti-metastatic or anti-cancer activity, said method comprising
administering said substance to a model obtained by the method claimed in claim 1;
allowing the cancer cell lines to metastasize for a suitable time period; and
determining anti-metastatic or anti-cancer activity by analogy with a control model.

20. The method as claimed in claim 19, wherein said anti-metastatic or anti-cancer activity is assessed by measuring the inhibitory or preventive effect of said substance on metastasis.

21. The method as claimed in claim 19, wherein said anti-metastatic or anti-cancer activity is determined by luciferase assisted visual imaging.

22. The method as claimed in claim 19, wherein said suitable time is at least a period in the range of 1 to 6 weeks.

23. The method as claimed in claim 19, wherein said metastasis is liver metastasis of colorectal cancer.

24. A method for evaluating efficiency of a treatment against metastasis of colorectal cancer, comprising:
administering the treatment to a model obtained by the method claimed in claim 1;
allowing the cancer cells to metastasize for a suitable time period; and
determining anti-metastatic or anti-cancer activity of said treatment by analogy with a control model.

25. The method as claimed in claim 24, wherein said suitable time is at least a period in the range of 1 to 6 weeks.

26. The method as claimed in claim 24, wherein said metastasis is liver metastasis of colorectal cancer.

27. The method as claimed in claim 24, wherein said anti-metastatic or anti-cancer activity is determined by luciferase assisted visual imaging.

28. The method as claimed in claim 24, wherein said anti-metastatic or anti-cancer activity is assessed by measuring the inhibitory or preventive effect of said substance on metastasis.


SEQUENCE LISTING (4 sequences)

SEQ ID NO: 1 - DNA, 1696 bp, Homo sapiens
  source 1..1696: mol_type = genomic DNA
cagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagacgaggacagtgggaaaggctcccagccccccagcccaccctcaccggctccttcgtccttctcctctacttcagcctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacactgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccgtggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacccccccccacaaggaaccctcaggccaccctccacgaggtgtgactaactatgcaataatccaccccaggtgcagccccagggcctgcggaggcggtggcagactagagtttagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaaaatgtttacatttttaaaggcacactggtatttatatttcaaacattttgtatcaaggaaacgttttgtatagttatatgtacagtttattgatattcaataaagcagttaatttatatattaaaaaaaaaaaaaaaaa

SEQ ID NO: 2 - DNA, 797 bp, Homo sapiens
  source 1..797: mol_type = genomic DNA
gaggcgccccgctcttctcctctgccccgggcccgcgaggccacgcgtcgccgctcgagagatgatgcaggacgtgtccagctcgccagtctcgccggccgacgacagcctgagcaacagcgaggaagagccagaccggcagcagccgccgagcggcaagcgcgggggacgcaagcggcgcagcagcaggcgcagcgcgggcggcggcgcggggcccggcggagccgcgggtgggggcgtcggaggcggcgacgagccgggcagcccggcccagggcaagcgcggcaagaagtctgcgggctgtggcggcggcggcggcgcgggcggcggcggcggcagcagcagcggcggcgggagtccgcagtcttacgaggagctgcagacgcagcgggtcatggccaacgtgcgggagcgccagcgcacccagtcgctgaacgaggcgttcgccgcgctgcggaagatcatccccacgctgccctcggacaagctgagcaagattcagaccctcaagctggcggccaggtacatcgacttcctctaccaggtcctccagagcgacgagctggactccaagatggcaagctgcagctatgtggctcacgagcggctcagctacgccttctcggtctggaggatggagggggcctggtccatgtccgcgtcccactagcaggcggagccccccaccccctcagcagggccggagacctaggtaaggaccgcgccgctgcaccccttcgcctctcaggtggcagacggcaggccggccaggccgcggttcccagtccacctcgatt

SEQ ID NO: 3 - DNA, 126 bp, Homo sapiens
  source 1..126: mol_type = genomic DNA
aatttatgcaataagacctattctacgttctctgggctggccaaacacaagcagctgcactgtgatgaccagtctgggaaatcggtcanctgcaagttctgtgacaaggaatatgtgagcctgggt

SEQ ID NO: 4 - DNA, 3466 bp, Homo sapiens
  source 1..3466: mol_type = genomic DNA
atttagacacaagcgagaggatcatggcggatggccccaggtgtaagcgcagaaagcgggcgaacccgcggcgcaataacgttacaaattataatactgtggtagaaacaaattcagattcagatgatgaagacaaactgcatattgtggaagaagaaagtgttacagatgcagctgactgtgaaggtgtaccagaggatgacctgccaacagaccagacagtgttaccagggaggagcagtgaaagagaagggaatgctaagagctgctggaaggatgacataaaagatgatgaatgcgagtcagatgcagaaaatgagcaaaaccatgatcctaatgttgaagagtttctacaacaacaagacactgctgtcatttttcctgaggcacctgaagaggaccagaggcagggcacaccagaagccagtggtcatgatgaaaatggaacaccagatgcattttcacaattactcacctgtccatattgtgatagaggctataaacgctttacctctctgaaagaacacattaaatatcgtcatgaaaagaatgaagataactttagttgctccctgtgcagttacacctttgcatacagaacccaacttgaacgtcacatgacatcacataaatcaggaagagatcaaagacatgtgacgcagtctgggtgtaatcgtaaattcaaatgcactgagtgtggaaaagctttcaaatacaaacatcacctaaaagagcacttaagaattcacagtggagagaagccatatgaatgcccaaactgcaagaaacgcttttcccattctggctcctatagctcacacataagcagtaagaaatgtatcagcttgatacctgtgaatgggcgaccaagaacaggactcaagacatctcagtgttcttcaccgtctctttcagcatcaccaggcagtcccacacgaccacagatacggcaaaagatagagaataaaccccttcaagaacaactttctgttaaccaaattaaaactgaacctgtggattatgaattcaaacccatagtggttgcttcaggaatcaactgttcaacccctttacaaaatggggttttcactggtggtggcccattacaggcaaccagttctcctcagggcatggtgcaagctgttgttctgccaacagttggtttggtgtctcccataagtatcaatttaagtgatattcagaatgtacttaaagtggcggtagatggtaatgtaataaggcaagtgttggagaataatcaagccaatcttgcatccaaagaacaagaaacaatcaatgcttcacccatacaacaaggtggccattctgttatttcagccatcagtcttcctttggttgatcaagatggaacaaccaaaattatcatcaactacagtcttgagcagcctagccaacttcaagttgttcctcaaaatttaaaaaaagaaaatccagtcgctacaaacagttgtaaaagtgaaaagttaccagaagatcttactgttaagtctgagaaggacaaaagctttgaagggggggtgaatgatagcacttgtcttctgtgtgatgattgtccaggagatattaatgcacttccagaattaaagcactatgacctaaagcagcctactcagcctcctccactccctgcagcagaagctgagaagcctgagtcctctgtttcatcagctactggggatggcaatttgtctcctagtcagccacctttaaagaacctcttgtctctcctaaaagcatattatgctttgaatgcacaaccaagtgcagaagagctctcaaaaattgctgattcagtaaacctaccactggatgtagtaaaaaagtggtttgaaaagatgcaagctggacagatttcagtgcagtcttctgaaccatcttctcctgaaccaggcaaagtaaatatccctgccaagaacaatgatcagcctcaatctgcaaatgcaaatgaaccccaggacagcacagtaaatctacaaagtcctttgaagatgactaactccccagttttaccagtgggatcaaccaccaatggttccagaagtagtacaccatccccatcacctctaaacctttcctcatccagaaatacacagggttacttgtacacagctgagggtgcacaagaagagccacaagtagaacctcttgatctttcactaccaaagcaacagggagaattattagaaaggtcaactatcactagtgtttaccagaacagtgtttattctgtccaggaagaacccttgaacttgtcttgcgcaaaaaaggagccacaaaaggacagttgtgttacagactcagaaccagttgtaaatgtaatcccaccaagtgccaaccccataaatatcgctatacctacagtcactgcccagttacccacaatcgtggccattgctgaccagaacagtgttccatgcttaagagcgctagctgccaataagcaaacgattctgactccccaggtggcatacacctactcaactacggtcagccctgcagtccaagaaccacccttgaaagtgatccagccaaatggaaatcaggatgaaagacaagatactagctcagaaggagtatcaaatgtagaggatcagaatgactctgattctacaccgcccaaaaagaaaatgcggaagacagaaaatggaatgtatgcttgtgatttgtgtgacaagatattccaaaagagtagttcattattgagacataaatatgaacacacaggtaaaagacctcatgagtgtggaatctgtaaaaaggcatttaaacacaaacatcatttgattgaacacatgcgattacattctggagaaaagccctatcaatgtgacaaatgtggaaagcgcttctcacactctgggtcttattctcaacacatgaatcatcgctactcctactgtaagagagaagcggaagaacgtgacagcacagagcaggaagaggcagggcctgaaatcctctcgaatgagcacgtgggtgccagggcgtctccctcacagggcgactcggacgagagagagagtttgacaagggaagaggatgaagacagtgaaaaagaggaagaggaggaggataaagagatggaagaattgcaggaagaaaaagaatgtgaaaaaccacaaggggatgaggaagagggggaggaggaggaagaagtggaagaagaagaggtagaagaggcagagaatgagggagaagaagcaaaaactgaaggtctgatgaaggatgacagggctgaaagtcaagcaagcagcttaggacaaaaagtaggcgagagtagtgagcaagtgtctgaagaaaagacaaatgaagcctaatcgtttttctagaaggaaaataaattctaattgataatgaatttcgttcaatattatccttgcttttcatggaaacacagtaacctgtatgctgtgattcctgttcactactgtgtaaagtaaaaact